(19) United States
(12) Patent Application Publication
(10) Pub. No.: US 2006/0218854 A1
(43) Pub. Date: Oct. 5, 2006

(54) Fuel Additive Composition and Fuel Composition and Method Thereof

(76) Inventors: Robert H. Barbour, Ashbourne (GB); David A. Duncan, Near Ashbourne (GB); David J. Moreton, Belper (GB)

Correspondence Address: THE LUBRIZOL CORPORATION;ATTN: DOCKET CLERK, PATENT DEPT., 29400 LAKELAND BLVD., WICKLIFFE, OH, 44092, US

(21) Appl. No.: 11/424,068

(22) Filed: Jun. 14, 2006

Related U.S. Application Data
(63) Continuation of application No. 10/470,426, filed on Jul. 28, 2003, now abandoned.
(60) Provisional application No. 60/268,733, filed on Feb. 14, 2001.

(51) Int. Cl. C10L 1/00; C07D 207/40

(52) U.S. Cl. 44/331; 44/418; 548/545

(57) ABSTRACT

A fuel additive composition includes a reaction product of a succinic acylating agent and a polyamine having at least one condensable primary amine group where the reaction product has a ratio of the imide to amide infrared carbonyl absorption peak areas of about 1:0.0-0.6 and a water content of about 0.3% or less by weight. The succinic acylating agent is prepared by thermal condensation of a highly reactive polyolefin with maleic anhydride or a reactive equivalent thereof. This fuel additive composition has a low chlorine content and is very effective in fuel compositions that include a normally liquid fuel in reducing deposits in the fuel intake system of an internal combustion engine.

### BACKGROUND OF THE INVENTION

1. Field of the Invention.

This invention involves fuel additive compositions, fuel compositions that include the fuel additive compositions, and a method of operating internal combustion engines with the fuel compositions. The compositions of the present invention are effective in reducing deposits in fuel intake systems of internal combustion engines.

2. Description of the Related Art.

Deposits form in the fuel intake system of an internal combustion engine and can adversely affect combustion performance in terms of power output and emissions. Fuel additives are generally incorporated into a fuel to reduce deposits and maintain satisfactory engine performance. Fuel additives that are improved in reducing deposits are advantageous.

Hydrocarbon substituted succinimides, derived from a succinic acylating agent which is prepared by thermal condensation of a highly reactive polyisobutylene with maleic anhydride or a reactive equivalent thereof, provide deposit reduction performance in fuel compositions and have a low chlorine content which is an environmental feature now required in Europe.

U.S. Application Serial No. 60/219,791 filed Jul. 19, 2000 (Duncan et al.) disclose an additive composition comprising the combination of a succinimide and an oil, having a viscosity of 100 to 400 centistokes at 40° C., that can reduce fouling of middle distillate fuel oil injectors in a compression ignition engine.

International Publication No. WO 99/32585 published Jul. 1, 1999 (Moreton) discloses polyisobutene substituted succinimides derived from N-aminoalkyl substituted imidazoles that are detergents for use in fuels.

U.S. Pat. No. 5,925,151 filed Sep. 19, 1996 (DeCanio et al.) disclose a detergent additive composition comprising the combination of a monosuccinimide, derived from polyisobutylene and a polyethylene polyamine, and an aromatic hydrocarbon diluent that may be used in diesel fuel to remove or prevent deposits.

It has now been found that the succinimides of the present invention, prepared by thermal condensation of a highly reactive polyolefin with maleic anhydride or a reactive equivalent thereof followed by amination of the thermal condensate with a polyamine under conditions that increase imide content and decrease water content, provide improved performance in fuel compositions in reducing deposits in fuel intake systems of internal combustion engines. The succinimides of the present invention also have the environmental feature of a low chlorine content.

### SUMMARY OF THE INVENTION

It is an object of this invention to reduce deposits in the fuel intake system of an internal combustion engine fueled with a normally liquid fuel.

It is a further object of this invention to reduce deposits in the fuel intake system of a compression ignition engine fueled with a diesel fuel.

The objects, advantages and embodiments of the present invention are in part described in the specification and in part are obvious from the specification or from the practice of the invention. Therefore, it is understood that the invention is covered as described or obvious that falls within the scope of the appended claims.

To achieve the foregoing objects in accordance with the invention as described and claimed herein, the fuel additive composition of the present invention comprises a reaction product of an aliphatic hydrocarbon substituted succinic acylating agent and a polyamine having at least one condensable primary amine group represented by the formula —NH₂ wherein said reaction product has a ratio of the imide to amide infrared carbonyl absorption peak areas of about 1:0.0-0.6 and a water content of about 0.3% or less by weight, and wherein the aliphatic hydrocarbon substituted succinic acylating agent is prepared by thermal condensation of a highly reactive polyolefin with maleic anhydride or a reactive equivalent thereof.

A further embodiment of the present invention is a fuel composition comprising a major amount of a normally liquid fuel that is a gasoline or a diesel fuel; and the above-described fuel additive composition.

Another aspect of the present invention is a method of reducing deposits in the fuel intake system of an internal combustion engine by operating the engine with the above-described fuel composition.

### DETAILED DESCRIPTION OF THE INVENTION

The fuel additive compositions of the present invention comprise a reaction product of an aliphatic hydrocarbon substituted succinic acylating agent and polyamine.

The aliphatic hydrocarbon substituent of the succinic acylating agent can be a straight- or branched-chain hydrocarbon group, and can be saturated, olefinic with one or more carbon-to-carbon double bonds, or acetylenic with a carbon-to-carbon triple bond. The aliphatic hydrocarbon substituent is usually a branched-chain, olefinic group.

The polyamine contains two or more amine groups where at least one of the amine groups is a condensable primary amine group represented by the formula —NH₂.

The reaction product of the present invention of the succinic acylating agent and polyamine has a ratio of the imide to amide infrared carbonyl absorption peak areas of about 1:0.0-0.6 and a water content of about 0.3% or less by weight. The reaction of the succinic acylating agent and polyamine can result in a reaction product that contains several components including the succinate derivatives imide, amide and ammonium carboxylate salt. The reaction of the succinic acylating agent and polyamine is run under conditions detailed herein that increases the amount of the imide relative to the amide and ammonium carboxylate salt and that decreases the water content of the reaction product.

The succinic acylating of the present invention is usually a succinic anhydride, but can be a reactive equivalent to include the bisacid, bisesters, and acid-esters and bis acyl halides. The succinic acylating agent is prepared by a thermal condensation of a highly reactive polyolefin with maleic anhydride or a reactive equivalent thereof to include maleic acid, bis acyl halides, bisesters and acid-esters. Maleic anhydride is generally used.

The highly reactive polyolefin used to prepare the succinic acylating agent of the present invention contains a major amount of its carbon to carbon double bonds in a terminal position on the carbon chain as vinylidene type double bonds represented by the formula 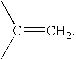
In one embodiment at least 70% of the double bonds in the highly reactive polyolefin are vinylidene type, in another instance at least 80% are vinylidene type, and in yet another instance at least 90% are vinylidene type.

The highly reactive polyolefin can contain about 30 to about 180 carbon atoms, and in another instance about 40 to about 130 carbon atoms. The polyolefin usually contains about 55 to about 90 carbon atoms.

The highly reactive polyolefin can be derived from homopolymerized or interpolymerized 1-olefins having 2 to 18 carbon atoms. The 1-olefins include ethylene, propylene, the butene isomers 1-butene, 2-butene and isobutylene, and mixtures of 1-olefins. Examples of polyolefins are polyethylenes, polypropylenes, polyisobutylenes, especially those derived from refinery streams, copolymers of ethylene and 1-olefins such as ethylene-propylene copolymers, and terpolymers of ethylene with 1 -olefins and dienes such as ethylene-propylene-diene terpolymers.

A preferred highly reactive polyolefin is a polyisobutylene having a high vinylidene content of 70% or more. The polyisobutylene can have a number average molecular weight of about 400 to about 2,500, while about 600 to about 1,800 is more preferred, and about 800 to about 1,200 is most preferred. Well known procedures involving gel phase chromatography and vapor phase osmometry are used to determine the number average molecular weights of polyolefins such as polyisobutylene. Polyisobutylene having a high vinylidene content is commercially available from BASF under the tradename Glissopal®.

The succinic acylating agents of the present invention are prepared by thermally condensing a highly reactive polyolefin with maleic anhydride or a reactive equivalent thereof. Procedures for the thermal condensation are well known and usually involve temperatures above 200° C. at an elevated pressure. U.S. Application Serial No. 60/219,791 filed Jul. 19, 2000 (Duncan et al.) describe the procedure for a thermal condensation of a polyisobutylene having a high vinylidene content with maleic anhydride, the disclosure of which is incorporated herein by reference. There is a two-fold advantage to preparing the succinic acylating agent via a thermal condensation with a highly reactive polyolefin. First, the thermal condensation provides a nonchlorine route to the succinic acylating agents so that their succinimide derivatives have a low chlorine content. Second, conversion of the highly reactive polyolefins to hydrocarbon substituted succinic acylating agents is high so that their succinimide derivatives have a high active chemical content and are effective as fuel additives in reducing deposits in fuel intake systems.

The polyamines of the present invention include polyethylene polyamines as well as other types of polyamines. Polyethylene polyamines are available from Dow and Union Carbide and include ethylenediamine, diethylenetriamine, triethylenetetramine, tetraethylenepentamine, pentaethylenehexamine and polyethylene polyamine bottoms. Other types of polyamines include polyalkylene polyamines in addition to polyethylene polyamines described above, heterocyclic polyamines, aromatic heterocyclic polyamines, polyamines containing one or more hydroxyl groups and ether or polyether containing polyamines. Examples of other types of polyamines are tris(2-aminoethyl)amine, propylenediamine, dipropylenetriamine, N-(2-aminoethyl)-1,3-propanediamine, N,N′-bis(2-aminoethyl)-1,3-propanediamine, dimethylaminopropylamine, diethylaminopropylamine, 1-(3-aminopropyl)imidazole and 4-(3-aminopropyl)morpholine.

The succinic acylating agent and polyamine of the present invention can be reacted using various molar ratios of the two reactants. A preferred molar ratio of succinic acylating agent to polyamine is about 1:0.3-1.1, more preferred is about 1:0.5-1, and most preferred is about 1:0.74-1.

The present invention involves the reaction product of a succinic acylating agent, derived from thermally condensing a highly reactive polyolefin and maleic anhydride or a reactive equivalent thereof, and a polyamine under reaction conditions wherein the reaction product has an increased imide content and decreased water content relative to analogous materials prepared under conditions less selective for increased imide and decreased water content. It has been found that increasing the level of imide relative to amide and ammonium carboxylate salt in the reaction product provides a fuel additive composition with improved deposit reduction performance in the fuel intake system of an internal combustion engine. Additionally, it has been found that decreasing the level of water in the reaction product provides a fuel additive composition that is much more stable and retains a nearly constant imide level for a long period of time. In contrast, analogous materials prepared under conditions less selective for increased imide level and decreased water level provide a reduced level of deposit reduction as fuel additives and also show a much greater decrease in imide content with time. The additive composition of the present invention provides improved deposit reduction performance due to the increased imide content. Additionally, this improved performance is deliverable even after a lengthy storage period due to the decreased water content which results in a more stable imide content with time.

The reaction products of the present invention have an increased level of imide relative to amide and ammonium carboxylate salt compared to analogous materials prepared under conditions less favorable to forming imide such as current commercial materials. The reaction products of the present invention have a ratio of the imide to amide to ammonium carboxylate salt infrared carbonyl absorption peak areas of about 1:0.0-0.6:0.0-0.3, more preferably of about 1:0.0-0.4:0.0-0.2, and most preferably of about 1:0.0-0.3:0.0-0.1. The infrared carbonyl absorption peak areas were obtained using a Perkin-Elmer “Spectrum One” program for imide at about 1,705 cm⁻¹, for amide at about 1,660 cm⁻¹ and for ammonium carboxylate salt at about 1,550 cm⁻¹. Samples of an analogous commercial material prepared under conditions less selective for imide formation had a ratio of the imide to amide to ammonium carboxylate salt infrared carbonyl absorption peak areas of about 1:0.8-1.4:0.3-0.5. This commercial material also provided less deposit reduction performance compared to reaction products of the present invention as detailed in examples that follow herein.

The reaction products of the present invention have a decreased level of water that provides a fuel additive composition that is more stable in having a nearly constant level of imide for several months. This enhanced stability of the imide level ensures delivery of improved performance from the increased level of imide even after a lengthy storage period. It is known that current commercial succinimide materials, prepared under less rigorous conditions for water removal, will over time decrease in imide content and increase in amide and ammonium carboxylate salt content. This decrease in imide content represents a loss in deposit reduction performance since improved performance has been found to be directly related to increased imide content. It has been found that the reaction products of the present invention have a nearly constant level of imide for about 3 months with the imide level only slowly decreasing over extended periods when the water content of the reaction products as determined by Karl Fischer titration is about 0.3% or less by weight, more preferably about 0.25% or less by weight, and most preferably about 0.2% or less by weight. Examples that follow illustrate the stability of the imide content when water content is decreased.

The reaction products of the succinic acylating agents and polyamines of the present invention are prepared under reaction conditions that result in products having a ratio of the imide to amide infrared carbonyl absorption peak areas of about 1:0.0-0.6 and a water content of about 0.3% or less by weight. The reaction conditions that can increase imide content and decrease water content are temperatures of about 140° C. or higher for an extended period of time, use of an inert gas sparge, use of reduced pressure, use of a solvent that forms an azeotrope with water, and combinations of two or more thereof. The following examples are illustrative of reaction conditions that result in reaction products of the present invention which have increased imide content and decreased water content.

### EXAMPLE A (COMPARATIVE)

A commercial succinimide, Komad 303 available from MOL of Hungary, has a ratio of imide to amide to ammonium carboxylate salt infrared carbonyl absorption peak areas of 1:1.39:0.5. Komad 303 is 60% active chemical in a 350 neutral mineral oil. Komad 303 is prepared by thermal condensation of a high vinylidene 1,000 molecular weight polyisobutene (BASF Glissopal® 1000) and a maleic anhydride followed by amination with tetraethylenepentamine in a molar ratio of about 1:0.87.

### EXAMPLE B (COMPARATIVE)

A commercial succinimide, Komad 303 from a batch different from that of Example A, has a ratio of imide to amide to ammonium carboxylate salt infrared carbonyl absorption peak areas of 1:0.78:0.3.

### EXAMPLE C

A sample (1003.5 g) of the commercial succinimide of Example A was placed in a flask fitted for distillation and heated with stirring to 180° C. Some foaming was observed. A vacuum was gradually applied, and the sample was held at 180° and a reduced pressure of 49mm Hg for 1 hour. The product residue weighed 986.1 g, and the distillate was a yellow liquid weighing 7.25 g. The product had a nitrogen content of 2.71%, a total base number of 74 (where base equivalents are expressed in terms of mg of KOH per g of sample), and a ratio of imide to amide to ammonium carboxylate salt infrared carbonyl absorption peak areas of 1:0.46:0.09.

### EXAMPLE D

A sample (300 g) of the commercial succinimide of Example B was placed in a flask fitted for distillation, heated with stirring to 175° C., and held at 175° C. for 3 hours. The sample was heated to 200° C., and 60 ml of 2-ethyl-1-hexanol was added. A vacuum was applied, and the sample and alcohol were held at 200° C. and a reduced pressure of 49 mm Hg for 1 hour. The product residue weighed 286.5 g. The distillate collected was 60 ml of the alcohol and 3 ml of water. The product had a nitrogen content of 2.51%, a total base number of 60 (as mg KOH per g of sample), a water content of 0.05%, and a ratio of imide to amide to ammonium carboxylate salt infrared carbonyl absorption peak areas of 1:0.21:0.02.

### EXAMPLE E

A sample (3.5 Kg) of an unfiltered polyisobutenylsuccinic anhydride, derived from a thermal condensation of a high vinylidene 1,000 molecular weight polyisobutene (BASF Glissopal® 1000) and maleic anhydride, and 2.333 Kg of a 330 neutral mineral oil were blended together at 50° C. for 0.5 hour. The blend was then filtered through diatomaceous earth. A sample of the filtrate (5 Kg) was placed in a flask fitted for distillation and heated with stirring under a nitrogen purge to 175° C. Tetraethylenepentamine (567.9 g at a molar ratio of succinic anhydride to polyamine of 1:0.87) was added dropwise over 95 minutes. The reaction mixture was held at 175° C. for 3 hours. Vacuum was applied, and the mixture was held at 175° C. and a reduced pressure of 49 mm Hg for 0.5 hour. Next 580 ml of 2-ethyl-1-hexanol was added, and the mixture was heated to 190° C. and held at 190° C. for 2.5 hours. A vacuum was applied, and the mixture held at 190° C. and a reduced pressure of 49 mm Hg to remove the alcohol and additional water. The distillate consisted of 580 ml of the alcohol and 18 ml of water. The product residue had a nitrogen content of 3.0%, a total base number of 83 (as mg KOH per g of sample), a water content of 0.25%, and a ratio of imide to amide to ammonium carboxylate salt infrared carbonyl absorption peak areas of 1:0.44:0.06. The product had a nearly constant imide level over a period of 98 days.

### EXAMPLE F

A sample (180 parts by weight) of an unfiltered polyisobutenylsuccinic anhydride, derived from a thermal condensation of a high vinylidene 1,000 molecular weight polyisobutene (BASF Glissopal® 1000) and maleic anhydride, and 120 parts by weight of a 330 neutral mineral oil were blended together at 50° C. for 0.5 hr. The blend was then filtered through diatomaceous earth. The filtrate was placed in a reactor fitted for distillation and heated with stirring to 110° C. Tetraethylenepentamine (31.8 parts at a molar ratio of succinic anhydride to polyamine of 1:0.72) was added over 3 hours at 110-120° C. The reaction mixture was heated to 175° C. over 6 hours, and was nitrogen sparged at 175° C. for 4 hours. The aqueous distillate was 4.6 parts. The product residue was 320.6 parts. The product had a nitrogen content of 3.36%, a total base number of 85.5 (as mg KOH per g of sample), a water content of 0.1%, and a ratio of imide to amide to ammonium carboxylate salt infrared carbonyl absorption peak areas of 1:0.23:0.03. The product had a nearly constant imide level over a period of 70 days.

### EXAMPLE G

A sample (180 g) of an unfiltered polyisobutenylsuccinic anhydride, derived from a thermal condensation of a high vinylidene 1,000 molecular weight polyisobutene (BASF Glissopal® 1000) and maleic anhydride, and 120 g of Solvesso 150 (high boiling aromatic solvent from Exxon Chemicals) were blended together for 0.5 hour. The blend was filtered through diatomaceous earth. The filtrate was placed in a flask fitted for distillation, and heated with stirring to 175° C. Tetraethylenepentamine (34.1 g at a molar ratio of succinic anhydride to polyamine of 1 :0.87) was added dropwise, and the reaction mixture was held at 175° C. for 4 hours. The product residue was 275 g. The distillate was 5.8 ml of a yellow liquid. The product had a nitrogen content of 3.83%, a total base number of 124 (as mg KOH per g of sample), a water content of 0.19%, and a ratio of imide to amide to ammonium carboxylate salt infrared carbonyl absorption peak areas of 1:0.24:0.04.

The fuel additive compositions of the present invention can include a carrier fluid. Suitable carrier fluids are alkylphenols including alkoxylated alkylphenols, esters of carboxylic acids including polyol esters and vegetable oils, alkoxylated alcohols including polyols, polyalkylene glycols, and mineral oils. Mineral oils having a viscosity ranging from about 100 to about 400 centistokes at 40° C. are especially useful.

In another embodiment of the present invention, the fuel additive compositions can include a solvent. Suitable solvents are aliphatic hydrocarbons, aromatic hydrocarbons, glycol ethers, alcohols, and mixtures thereof.

The fuel additive compositions in a further embodiment of the present invention can include additional fuel additives such as cold flow improvers, pour point depressants, storage stabilizers, corrosion inhibitors such as alkenylsuccinic acids, cetane improvers, anti-knock additives, anti-static agents, biocidal additives, smoke suppressants, other types of detergents, antifoam agents including silicone fluids, lubricity additives such as tall oil fatty acid, and demulsifiers such as alkoxylated alkylphenols.

Another embodiment of the present invention is a fuel composition comprising a major amount of a normally liquid fuel and the fuel additive composition of the present invention. The normally liquid fuel can be a gasoline or a diesel fuel. The gasoline can be one or more hydrocarbon fractions generally boiling in the range from 30° C to 230° C. The gasoline can contain oxygenates, oxygen-containing compounds, including alcohols such as ethanol and ethers such as methyl t-butyl ether. The diesel fuel can be a hydrocarbon diesel fuel from one or more hydrocarbon fractions generally boiling in the range from 140° C. to 400° C., for example, from 170 to 350° C. The hydrocarbon diesel fuel usually contains several hydrocarbon fractions. In one embodiment the hydrocarbon diesel fuel has at least 90% by volume hydrocarbon fraction boiling at 350° C., in a second embodiment greater than 95% by volume fraction boiling at 350° C., in a third embodiment at least 10% by volume fraction boiling at 180° C., and in a fourth embodiment at least 15% by volume fraction boiling at 180° C. The aromatic content of the hydrocarbon diesel fuel is typically less than 40% by volume, in another instance less than 30%, and in a further instance less than 20%. The cetane number of the hydrocarbon diesel fuel is usually greater than 40, in another embodiment greater than 45, and in a further embodiment greater than 50. The sulfur content of the hydrocarbon diesel fuel can be 0.5 wt % or less, in a second instance 0.2 wt % or less , and in a third instance 0.05 wt % or less, and in a fourth instance 0.0010 wt % or less. The diesel fuel can be a biodiesel fuel. The biodiesel fuel includes esters of naturally occurring fatty acids which can be prepared by the transesterification of triglycerides of natural fats and oils with lower aliphatic alcohols to include methyl, ethyl, propyl and butyl alcohol. Natural fats and oils include sunflower oil, rapeseed oil, coriander oil, castor oil, soybean oil, cottonseed oil, peanut oil, coconut oil, and beef tallow. The diesel fuel can be a mixture of a hydrocarbon diesel fuel and a biodiesel fuel such as the methyl ester of rapeseed oil. Fuel compositions of the present invention that include a diesel fuel are especially useful in compression ignition engines to reduce deposits in the fuel intake system.

Fuel compositions of the present invention include the reaction products of the succinic acylating agents and the polyamines at a level sufficient to provide deposit reduction performance. The level of the reaction product in the fuel composition can range from about 10 ppm to about 1,000 ppm based on the weight of the fuel composition, in another instance from about 20 ppm to about 600 ppm, and also from about 30 ppm to about 300 ppm.

The fuel additive compositions and fuel compositions of the present invention are prepared by mixing components at ambient temperatures or at somewhat elevated temperatures ranging from about 40° C. to 60° C. until the mixture is homogeneous. The components can include the reaction products of the succinic acylating agents and polyamines, carrier fluids, solvents, additional fuel additives, and normally liquid fuels.

A further embodiment of the present invention is a method of reducing deposits in the fuel intake system of an internal combustion engine by operating the engine with the fuel composition of the present invention. This method is especially effective in compression ignition engines fueled with diesel fuels in reducing deposits in and around fuel injector nozzles. This deposit reduction performance includes both preventing deposits from forming as well as removing or cleaning up deposits that have formed.

The following examples show the benefit of the present invention in providing improved deposit reduction performance in the fuel intake system of an internal combustion engine.

Peugeot XUD-9 Diesel Engine Test¹

| | Imide to Amide | | Average % Flow |
|---|---|---|---|
| Detergent² | Ratio³ | Treat, ppm⁴ | Remaining¹ |
| Example | 1:0.87 | 100 | 31 |
| A(comparative) | | | |
| Example C | 1:0.46 | 100 | 34 |
| Example | 1:0.78 | 80 | 28 |
| B(comparative) | | | |
| Example E | 1:0.44 | 80 | 38 |
| Example | 1:0.78 | 80 | 32 |
| B(comparative) | | | |
| Example D | 1:0.21 | 80 | 37 |
| Example F | 1:0.23 | 80 | 38 |
| Example G | 1:0.24 | 80 | 44 |
| Komad 303⁵ | 1:1.3 | 60⁷ | 32 |
| (comparative) | | (biodiesel) | |
| High Imide | 1:0.03 | 60⁷ | 36 |
| Succinimide⁶ | | (biodiesel) | |

¹The Peugeot XUD-9 diesel engine 10 hour test is used to evaluate the effectiveness of a detergent that is added to a diesel fuel as part of a diesel fuel additive package. The test evaluates deposit reduction performance of the detergent by measuring the average % flow remaining for the fuel injectors at the end of test. Detergents having larger % flow remaining values are more effective.
²The detergents are the succinimide compositions of Examples A-G that were described in detail earlier in the detailed description. Examples A and B are comparative while Examples C-G are embodiments of the present invention showing improved deposit reduction performance.
³Ratios are determined as the ratio of infrared carbonyl absorption peak areas for the imide at about 1,705 cm⁻¹ and for the amide at about 1,660 cm⁻¹ using a Perkin-Elmer “Spectrum One” program.
⁴The treat levels are active chemical levels of the detergents in the diesel fuel.
⁵Komad 303 is a commercial succinimide available from MOL of Hungary.
⁶The high imide succinimide was prepared by the procedure of Example F.
⁷The fuel consisted of 70% by volume of a standard hydrocarbon diesel fuel and 30% by volume of a biodiesel fuel which was the methyl ester of rapeseed oil.

Cummins L-10 Injector Deposit Test¹

| Detergent | Imide to Amide Ratio² | Deposit Rating³ |
|---|---|---|
| Komad 303⁴ | 1:0.78 | 12.5 |
| (comparative) | | |
| Komad 303⁴ | between 1:0.78 | 15.2 |
| (comparative) | and 1:1.3⁵ | |
| Example F | between 1:0.23 | 8.4 |
| | and 1:0.48⁵ | |
| ¹Cummins L-10 fuel injector deposit test uses two Cummins L-10 diesel engines mounted in tandem configuration with one engine operated to drive the second nonoperated engine in a test cycle capable of producing injector deposits. U.S. Pat. No. 6,042,626 further describes the procedure, and test details are available from Engineering Test Services, a division of the Cummins Engine Co., Charleston, S.C., U.S.A. In | | |
| # the test standard hydrocarbon diesel fuel was treated with each detergent at the same commercial treat level on an active chemical basis. | | |

²Ratios are determined as the ratio of infrared carbonyl absorption peak areas for imide at about 1,705 cm⁻¹ and for the amide at about 1,660 cm⁻¹ using a Perkins-Elmer “Spectrum One” program.
³An average deposit rating of 10 or lower for the fuel injectors is a good, desirable result.
⁴Komad 303 is a commercial succinimide available from MOL of Hungary.
⁵Infrared analyses of the detergent were available for dates that bracketed the test date.

What is claimed is:

1. A fuel additive composition, comprising:
a reaction product of an aliphatic hydrocarbon substituted succinic acylating agent and a polyamine having at least one condensable primary amine group represented by the formula —NH₂ wherein said reaction product has a ratio of the imide to amide infrared carbonyl absorption peak areas of about 1:0.0-0.6 and a water content of about 0.3% or less by weight, and wherein the aliphatic hydrocarbon substituted succinic acylating agent is prepared by thermal condensation of a highly reactive polyolefin with maleic anhydride or a reactive equivalent thereof.

2. The composition of claim 1 wherein the reaction product has a ratio of the imide to amide infrared carbonyl absorption peak areas of about 1:0.0-0.4 and a water content of about 0.25% or less by weight.

3. The composition of claim 1 wherein the reaction product has a ratio of the imide to amide infrared carbonyl absorption peak areas of about 1:0.0-0.3 and a water content of about 0.2% or less by weight.

4. The composition of claim 1 wherein the reactive polyolefin contains about 30 to about 180 carbon atoms and is derived from homopolymerized or interpolymerized 1-olefins having 2 to 18 carbon atoms.

5. The composition of claim 1 wherein the reactive polyolefin is a polyisobutylene having a number average molecular weight of about 400 to about 2,500.

6. The composition of claim 1 wherein the polyamine is selected from the group consisting of ethylenediamine, diethylenetriamine, triethylenetetramine, tetraethylenepentamine, pentaethylenehexamine, polyethylene polyamine bottoms, and mixtures of two or more thereof.

7. The composition of claim 1 wherein the polyamine is tetraethylenepentamine.

8. The composition of claim 6 wherein the reactive polyolefin is a polyisobutylene.

9. The composition of claim 8 wherein the molar ratio of the succinic acylating agent to the polyamine is about 1:0.5-1.

10. The composition of claim 1 further comprising a carrier fluid.

11. The composition of claim 10 wherein the carrier fluid is an oil having a viscosity of about 100 to about 400 centistokes at 40° C.

12. The composition of claim 1 further comprising a solvent.

13. The composition of claim 12 wherein the solvent is an aliphatic hydrocarbon, aromatic hydrocarbon or mixtures thereof.

14. A fuel composition comprising a major amount of a normally liquid fuel that is a gasoline or a diesel fuel; and the fuel additive composition of claim 1.

15. The fuel composition of claim 14 wherein the diesel fuel is a hydrocarbon diesel fuel, a biodiesel fuel, or a mixture thereof.

16. The fuel composition of claim 14 wherein the reaction product of the succinic acylating agent and the polyamine ranges from about 10 ppm to about 1,000 ppm based on the weight of the fuel composition.

17. A method of reducing deposits in the fuel intake system of an internal combustion engine by operating the engine with the fuel composition of claim 14.
